(19) United States
(12) Patent Application Publication
(10) Pub. No.: US 2009/0228045 A1
(43) Pub. Date: Sep. 10, 2009

(54) Dynamic rod

(76) Inventors: Stanley Kyle Hayes, Mission Viejo, CA (US); Joey Camia Reglos, Lake Forest, CA (US); Moti Altarac, Irvine, CA (US); Daniel H. Kim, Houston, TX (US)

Correspondence Address: RIMAS LUKAS;VERTIFLEX, INC., 1351 CALLE AVANZADO, SAN CLEMENTE, CA, 92673, US

(21) Appl. No.: 12/366,089

(22) Filed: Feb. 5, 2009

Related U.S. Application Data
(63) Continuation-in-part of application No. 12/233,212, filed on Sep. 18, 2008.
Application No. 12/233,212 is a continuation-in-part of application No. 12/154,540, filed on May 23, 2008.
Application No. 12/154,540 is a continuation-in-part of application No. 11/427,738, filed on Jun. 29, 2006.
Application No. 11/427,738 is a continuation-in-part of application No. 11/436,407, filed on May 17, 2006.
Application No. 11/436,407 is a continuation-in-part of application No. 11/033,452, filed on Jan. 10, 2005.
Application No. 11/033,452 is a continuation-in-part of application No. 11/006,495, filed on Dec. 6, 2004.
Application No. 11/006,495 is a continuation-in-part of application No. 10/970,366, filed on Oct. 20, 2004.
(60) Provisional application No. 61/063,878, filed on Feb. 6, 2008.
(60) Provisional application No. 60/994,899, filed on Sep. 21, 2007.
(60) Provisional application No. 60/931,811, filed on May 25, 2007.

(51) Int. Cl. A61B 17/70

(52) U.S. Cl. 606/257; 606/264; 606/278

(57) ABSTRACT

A dynamic rod implantable into a patient and connectable between two vertebral anchors in adjacent vertebral bodies is provided. The dynamic rod fixes the vertebral bodies together in a dynamic fashion providing immediate postoperative stability and support of the spine. The dynamic rod comprises a first rod portion and a second rod portion connected together. The dynamic rod further includes at least a one bias element configured to provide a bias force in response to deflection or translation of the first rod portion relative to the second rod portion. The dynamic rod includes a locking construct which advantageously enables the extension and/or angulation of one rod portion with respect to the other rod portion to be reversibly locked in position. The dynamic rod permits relative movement of the first and second rod portions allowing the rod to carry some of the natural flexion and extension moments of the spine.

### CROSS-REFERENCE TO RELATED APPLICATIONS

This application claims the benefit of and is a continuation-in-part of U.S. Provisional Patent Application Ser. No. 61/063,878 entitled “Dynamic rod” filed on Feb. 6, 2008 which is incorporated herein by reference in its entirety. This application is a continuation-in-part of U.S. patent application Ser. No. 12/233,212 entitled “Dynamic rod” filed on Sep. 18, 2008 incorporated herein by reference in its entirety which is a non-provisional of U.S. Provisional Patent Application Ser. No. 60/994,899 entitled “Dynamic rod” filed on Sep. 21, 2007 which is incorporated herein by reference in its entirety. This application also claims priority to and is a continuation-in-part of co-pending U.S. patent application Ser. No. 12/154,540 entitled “Dynamic rod” filed on May 23, 2008 which is a non-provisional of U.S. Provisional Patent Application Ser. No. 60/931,811 entitled “Dynamic rod” filed on May 25, 2007, all of which are hereby incorporated by reference in their entireties. This application also claims priority to and is a continuation-in-part of co-pending U.S. patent application Ser. No. 11/427,738 entitled “Systems and methods for stabilization of the bone structures” filed on Jun. 29, 2006 which is a contintuation-in-part of U.S. patent application Ser. No. 11/436,407 entitled “Systems and methods for stabilization of the bone structures” filed on May 17, 2006 which is a continuation-in-part of U.S. patent application Ser. No. 11/033,452 entitled “Systems and methods for stabilization of the bone structures” filed on Jan. 10, 2005 which is a continuation-in-part of U.S. patent application Ser. No. 11/006,495 entitled “Systems and methods for stabilization of the bone structures” filed on Dec. 6, 2004 which is a continuation-in-part of U.S. patent application Ser. No. 10/970,366 entitled “Systems and methods for stabilization of the bone structures” filed on Oct. 20, 2004. All of the above-referenced applications are each incorporated herein by reference in their entirety.

### BACKGROUND

Damage to the spine as a result of advancing age, disease, and injury, has been treated in many instances by fixation or stabilization of vertebrae. Conventional methods of spinal fixation utilize a rigid spinal fixation device to support an injured spinal vertebra relative to an adjacent vertebra and prevent movement of the injured vertebra relative to an adjacent vertebra. These conventional spinal fixation devices include anchor members for fixing to a series of vertebrae of the spine and at least one rigid link element designed to interconnect the anchor members. Typically, the anchor member is a screw and the rigid link element is a rod. The screw is configured to be inserted into the pedicle of a vertebra to a predetermined depth and angle. One end of the rigid link element is connected to an anchor inserted in the pedicle of the upper vertebra and the other end of the rod is connected to an anchor inserted in the pedicle of an adjacent lower vertebra. The rod ends are connected to the anchors via coupling constructs such that the adjacent vertebrae are supported and held apart in a relatively fixed position by the rods. Typically two rods and two pairs of anchors are installed each in the manner described above such that two rods are employed to fix two adjacent vertebrae, with one rod positioned on each side of adjacent vertebrae. Once the system has been assembled and fixed to a series of two or more vertebrae, it constitutes a rigid device preventing the vertebrae from moving relative to one another. This rigidity enables the devices to support all or part of the stresses instead of the stresses being born by the series of damaged vertebra.

While these conventional procedures and devices have been proven capable of providing reliable fixation of the spine, the resulting constructs typically provide a very high degree of rigidity to the operative levels of the spine resulting in decreased mobility of the patient. Unfortunately, this high degree of rigidity imparted to the spine by such devices can sometimes be excessive. Because the patient's fixed vertebrae are not allowed to move, the vertebrae located adjacent to, above or below, the series that has undergone such fixation tend to move more in order to compensate for the decreased mobility. As a result, a concentration of additional mechanical stresses is placed on these adjacent vertebral levels and a sharp discontinuity in the distribution of stresses along the spine can then arise between, for example, the last vertebra of the series and the first free vertebra. This increase in stress can accelerate degeneration of the vertebrae at these adjacent levels.

Sometimes, fixation accompanies a fusion procedure in which bone growth is encouraged to bridge the intervertebral body disc space to thereby fuse adjacent vertebrae together. Fusion involves removal of a damaged intervertebral disc and introduction of an interbody spacer along with bone graft material into the intervertebral disc space. In cases where fixation accompanies fusion, excessively rigid spinal fixation is not helpful to the promotion of the fusion process due to load shielding away from the fixed series. Without the stresses and strains, bone does not have loads to adapt to and as bone loads decrease, the bone becomes weaker. Thus, fixation devices that permit load sharing and assist the bone fusion process are desired in cases where fusion accompanies fixation.

Various improvements to fixation devices such as a link element having a dynamic central portion have been devised. These types of dynamic rods support part of the stresses and help relieve the vertebrae that are overtaxed by fixation. Some dynamic rods are designed to permit axial load transmission substantially along the vertical axis of the spine to prevent load shielding and promote the fusion process. Dynamic rods may also permit a bending moment to be partially transferred by the rod to the fixed series that would otherwise be completely born by vertebrae adjacent to the fixed series. Compression or extension springs can be coiled around the rod for the purpose of providing de-rotation forces as well as relative translational sliding movement along the vertical axis of the spine. Overall, the dynamic rod in the fixation system plays an important role in recreating the biomechanical organization of the functional unit made up of two fixed vertebrae together with the intervertebral disc. In some cases or over time, a doctor may determine that it is best for the patient to substitute a rigid rod for a dynamic one or vice versa. No device currently on the market allows for the change without replacing the already imlanted rod. The present invention advantageously provides the doctor with an option to convert the same rod from a dynamic one to a rigid one and vice versa through a unique reversible locking mechanism that may be engaged percutaneously in a minimially invasive manner.

In conclusion, conventional spinal fixation devices have not provided a comprehensive solution to the problems associated with curing spinal diseases in part due to the difficulty of creating a system that mimics a healthy functioning spinal unit. Hence, there is a need for an improved dynamic spinal fixation device that provides a desired level of flexibility to the fixed series of the spinal column, while also providing long-term durability and consistent stabilization of the spinal column.

### SUMMARY

According to one aspect of the invention, a dynamic rod, implantable in a spine, comprises a first rod portion having a first engaging portion at a first end and a second rod portion having a second engaging portion at a first end. The first and second rod portions are connected to each other at the first and second engaging portions such that the first rod portion and second rod portion are capable of relative motion. The dynamic rod further includes a lock configured to lock said relative motion. In one variation of the dynamic rod, the lock is reversible. Generally, a bias element such as a spring is disposed between the first rod portion and the second rod portion to bias the movement of one rod portion relative to the other rod portion. In one variation, the dynamic rod is configured such that the relative motion is angulation of the first rod portion relative to the second rod portion. In another variation, the dynamic rod is configured such that the relative motion is longitudinal translation of the first rod portion relative to the second rod portion. In another variation, the dynamic rod is configured such that the relative motion is angulation of the first rod portion relative to the second rod portion and longtudinal translation of the first rod portion relative to the second rod portion. In one variation, the lock includes a spacer movable to a locked position between the first and second rod portions to arrest said relative motion. In another variation, the lock includes a ramp portion configured to provide ramp surface for the spacer to move against into a locked position. In a further variation, the spacer and ramp portion are located in the first engaging portion and the second rod portion is nested inside the first engaging portion such that when in the locked position the ramp portion abuts the first end of the second rod portion and the spacer abuts the ramp portion. In another variation, the dynamic rod includes an aperture for percutaneously engaging said lock.

According to another aspect of the invention, a dynamic rod, implantable in a spine, comprises a first rod portion coupled to a second rod portion and configured such that movement of one rod portion with respect to the other rod portion is lockable in position by a lock. In one variation, the movement is longitudinal translation or angulation of one rod portion with respect to the other rod portion. In another variation, the movement of one rod portion with respect to the other rod portion is reversibly lockable in position by the lock. In another variation, the longitudinal translation of the first rod portion with respect to the second rod portion is lockable by the lock while permitting the angulation of the first rod portion with respect to the second rod portion. In another variation, the angulation of one rod portion with respect to the other rod portion is lockable whereas the relative longitudinal translation is permitted. In one variation, the distance of the first rod portion from the second rod portion is lockable at any location within the range of motion. In another variation, the rod is lockable in a position such that the first rod portion is fully extended from the second rod portion. In another variation the lock operates such that the rod is lockable in a position such that the first rod portion is angled with respect to the second rod portion. In another variation, the lock operates such that the rod is lockable in a position such that the first rod portion is fully compressed towards the second rod portion. In one variation, the lock comprises an element movable in a substantially transverse direction to the longitudinal axis of the rod to a locked position. In another variation, the dynamic rod includes a spring disposed between the first and second rod portions.

### BRIEF DESCRIPTION OF THE DRAWINGS

FIG. 1a illustrates a perspective view of two dynamic rods according to the present invention each spanning two bone anchors implanted in a spinal motion segment.

FIG. 1b illustrates a perspective view of two dynamic rods according to the present invention each spanning two bone anchors and two non-dynamic rods each spanning two bone anchors; the bone anchors being implanted in three adjacent vertebral bodies.

FIG. 2a illustrates a perspective view of a dynamic rod according to the present invention.

FIG. 2b illustrates a perspective exploded view of a dynamic rod according to the present invention.

FIG. 2c illustrates a perspective cross-sectional view of a dynamic rod according to the present invention.

FIG. 3a illustrates a cross-sectional view of a first rod portion of a dynamic rod according to the present invention.

FIG. 3b illustrates a perspective view of a first rod portion of a dynamic rod according to the present invention.

FIG. 4a illustrates a cross-sectional view of a second rod portion of a dynamic rod according to the present invention.

FIG. 4b illustrates a perspective view of a second rod portion of a dynamic rod according to the present invention.

FIG. 5a illustrates a cross-sectional view of a retainer of a dynamic rod according to the present invention.

FIG. 5b illustrates a perspective view of a retainer of a dynamic rod according to the present invention.

FIG. 6a illustrates a side view of a lock of a dynamic rod according to the present invention.

FIG. 6b illustrates a perspective view of a lock of a dynamic rod according to the present invention.

FIG. 7a illustrates a perspective view of a slide of a dynamic rod according to the present invention.

FIG. 7b illustrates a top view of a slide of a dynamic rod according to the present invention.

FIG. 7c illustrates a cross-sectional view taken along line B-B of FIG. 7b of a slide of a dynamic rod according to the present invention.

FIG. 8a illustrates a side cross-sectional view of a dynamic rod in a fully extended position according to the present invention.

FIG. 8b illustrates a side cross-sectional view of a dynamic rod in a fully compressed position according to the present invention.

FIG. 8c illustrates a side cross-sectional view of a dynamic rod with phantom depictions of polyaxial displacement of the second rod portion relative to the first rod portion according to the present invention.

FIG. 8d illustrates a side view of a dynamic rod with phantom depictions of polyaxial displacement of the second rod portion relative to the first rod portion with the second rod portion fully extended relative to the first rod portion according to the present invention.

FIG. 8e illustrates a side view of a dynamic rod with phantom depictions of polyaxial displacement of the second rod portion relative to the first rod portion with the second rod portion fully compressed relative to the first rod portion according to the present invention.

FIG. 9a illustrates a side cross-sectional view of a dynamic rod according to the present invention.

FIG. 9b illustrates a side cross-sectional view of a dynamic rod with a lock partially advanced according to the present invention.

FIG. 9c illustrates a side cross-sectional view of a dynamic rod with a lock fully advanced according to the present invention.

### DETAILED DESCRIPTION

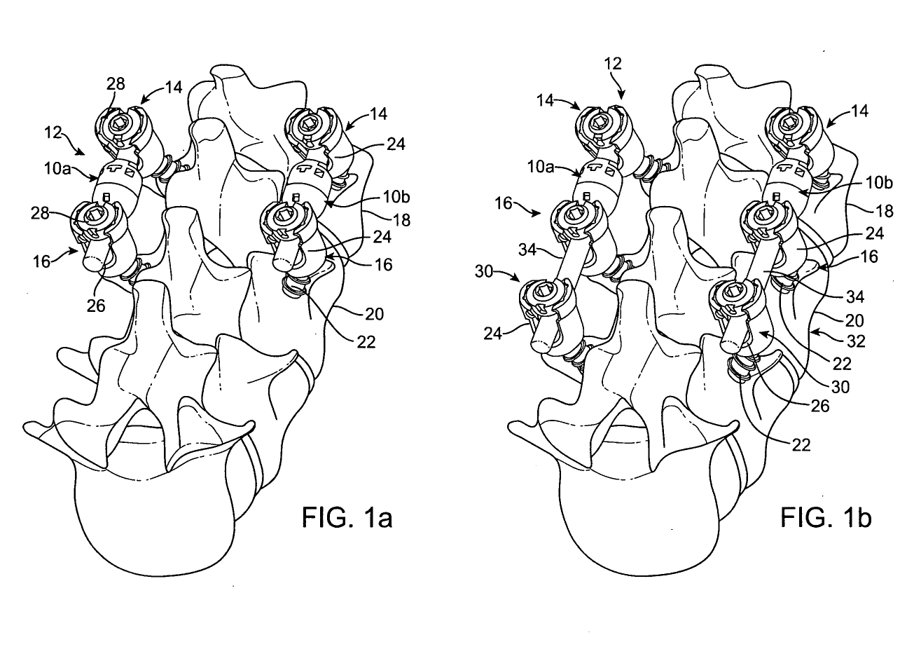

Referring now to FIGS. 1a and 1b, there is shown a dynamic rod 10a, 10b according to the invention for use in a spinal fixation system 12. A spinal fixation system 12 generally includes a first set 14 of two bone anchor systems installed into the pedicles of a superior vertebral segment 18, a second set 16 of two bone anchor systems installed into the pedicles of an inferior vertebral segment 20, a first link element 10a connected between one of the pedicle bone anchor systems in the first set and one of the pedicle bone anchor systems in the second set along the same side of the inferior and superior vertebral segments, and a second link element 10b connected between the other of the pedicle bone anchor systems in the first set and the other of the pedicle bone anchor systems in the second set along the same side of the inferior and superior vertebral segments.

A typical anchor system comprises, but is not limited to, a spinal bone screw 22 that is designed to have one end that inserts threadably into a vertebra and a seat 24 at the opposite end thereof. Typically, the seat 24 is designed to receive the link element 10a, 20b in a channel 26 in the seat 24. The link element 10a, 10b is typically a rod or rod-like member. The seat 24 typically has two upstanding arms that are on opposite sides of the channel that receives the rod member 10a, 10b. The rod 10a, 10b is laid in the open channel which is then closed with a closure member 28 to both capture the rod 10a, 10b in the channel 26 and lock it in the seat 24 to prevent relative movement between the seat 24 and the rod 10a, 10b. A multi-level installation is shown in FIG. 1b in which a third set 30 of two bone anchor systems are installed in the pedicles of a third vertebral segment 32. Non-dynamic link elements 34a, 34b are shown extending between the second set 16 and the third set 30 of bone anchor systems. The dynamic rod 10 of the present invention may be selectively employed by the surgeon in any multi-level, fully dynamic or semi-rigid spinal fixation system 12.

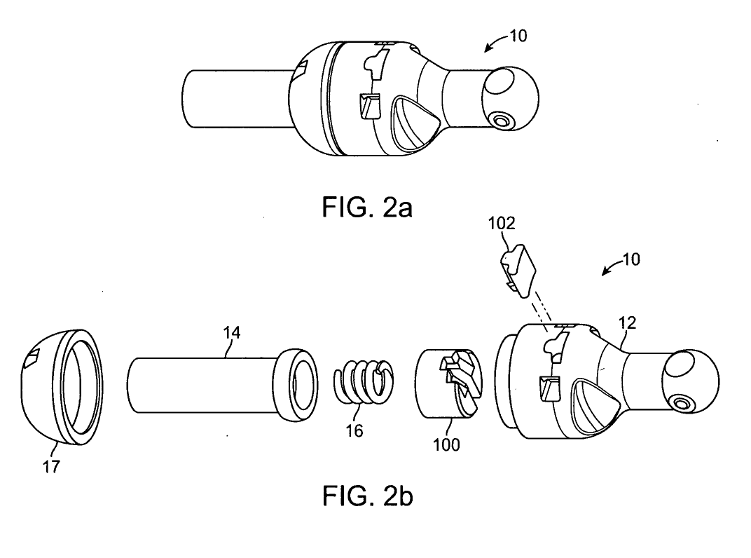

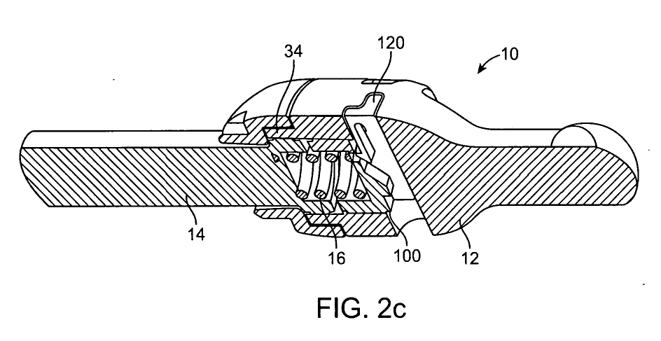

With particular reference to FIGS. 2a and 2b, a rod 10 according to the present invention comprises a first rod portion 12, a second rod portion 14, a bias element 16, a retainer 17 or other connecting means, a locking slide 100 and a dynamic lock or spacer 102. The first rod portion 12 is connected to the second rod portion 14 via the retainer 17. The locking slide 100 and the dynamic lock 102 are disposed inside the first rod portion 12 and the bias element 16 is disposed within and between the first and second rod portions 12, 14, and, in particular, the bias element 16 is disposed within the locking slide 100 as shown in FIG. 2c which illustrates a cross-section of the assembled rod 10.

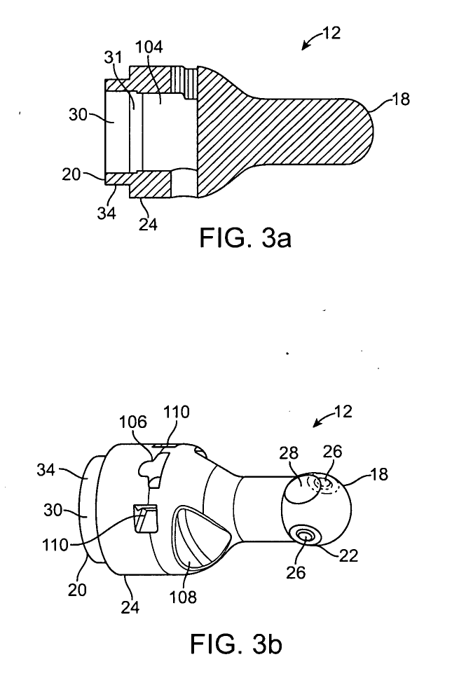

Referring now to FIGS. 3a and 3b, the first rod portion 12 of the dynamic rod 10 will now be described. The first rod portion 12 includes a first end 18 and a second end 20. The first rod portion 12 is generally cylindrical, elongate and rod-like in shape. An anchor connecting portion 22, shown in FIG. 3b, is formed at the first end 18 and configured for attachment to an anchor system. The anchor connecting portion 22 is partially spherical in shape and includes oppositely disposed outwardly extending pins 26 for engaging slots formed in the anchor to allow the dynamic rod 10 to pivot about the pins 26 when connected to the anchor. The anchor connecting portion 22 also includes oppositely disposed flat areas 28. When the dynamic rod 10 is connected to the anchor and pivoted into a substantially horizontal position, the flat areas 28 face upwardly and downwardly and as a result, provide a lower profile for the rod within the seat of the anchor. Furthermore, the flat areas 28 provide a flat contact surface for a closure member on the upper surface of the rod and a flat contact surface on the bottom surface when seated in the anchor. Although FIGS. 3a and 3b show the rod having an anchor connecting portion 22 configured for a pin-to-slot engagement, any suitable anchor connecting portion configuration is within the scope of the present invention.

Still referencing FIGS. 3a and 3b, the first rod portion 12 includes an engaging portion 24 at a slightly enlarged and bulbous second end 20. The engaging portion 24 is configured to engage the second rod portion 14 of the dynamic rod 10. The engaging portion 24 includes a first bore defining a receiving portion 30 for receiving the second rod portion 14. The engaging portion 24 also includes at least one abutment or ledge 31 formed within the first bore where there is a reduction in the bore diameter. The first bore also defines a locking slide receiving portion 104 configured for receiving the locking slide 100. The engaging portion 24 also includes a dynamic lock engaging aperture 106 and a dynamic lock release aperture 108 through the engaging portion 24 configured for accessing the dynamic lock 102 to engage or release it. The collar 34 has a slightly smaller outer diameter than the rest of the bulbous engaging portion 20. With the retainer 17 mated with the male member collar 34, the intersection of the first rod portion 12 and retainer 17 is flush. The outer surface of the first rod portion 12 further includes inserter notches 110 for an inserter instrument to grab the dynamic rod 10.

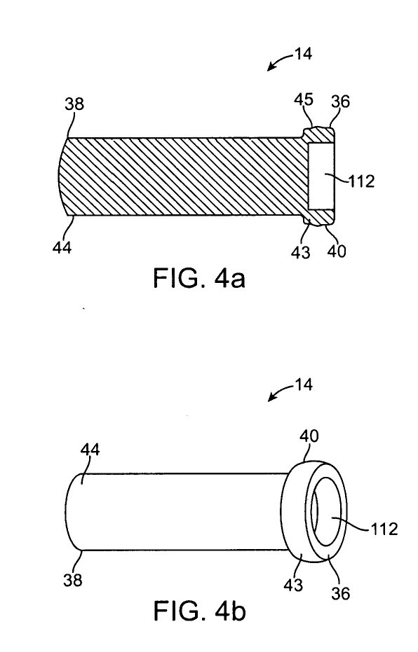

Turning now to FIGS. 4a and 4b, there is shown a second rod portion 14. The second rod portion 14 includes a first end 36 and a second end 38. The second rod portion 14 is generally cylindrical, elongate and rod-like in shape and includes an engaging portion 40 at the first end 36. The engaging portion 40 is configured to engage with the first rod portion 12 of the dynamic rod 10. The engaging portion 40 of the second rod portion 14 includes a spherical feature or collar 43 that allows the second rod portion 14 to angulate when placed inside the first rod portion 12. The first end 36 is shaped to form at least one abutment surface 45 (FIG. 4a) on the spherical collar 43 for contacting the receiving portion 30 wall of the first rod portion 12. At least a portion of the engaging portion 40 of the second rod portion 14 is configured and sized to fit within the receiving portion 30 of the first rod portion 14.

The second rod portion 14 further includes a bore opening at the first end 36 defining a bias element receiving portion 112 configured and sized to receive at least a portion of the bias element 16 therein.

Still referencing FIGS. 4a and 4b, the second end 38 of the second rod portion 14 includes an anchor connecting portion 44 configured to be connected to an anchor.

The anchor connecting portion 44 is sized and configured to be seated in a channel of a seat of a bone screw anchor for example. Any configuration for the second end 38 that is suitable for connection to an anchor is within the scope of the present invention and, for example, may include a rotatable pin-and-slot or other configuration similar to that shown in FIG. 3b.

Referring back to FIG. 2b, there is shown a bias element 16 according to the present invention. In the variation shown, the bias element 16 is a coil or spring. The bias element 16 is made from any suitable material such as surgical steel, titanium or PEEK. The bias element 16 is sized to be received inside the bias element receiving portion 112 and inside the locking slide 100 between the first rod portion 12 and the second rod portion 14. In one variation, a coiled spring is employed. In another variation, any suitable type of effective bias element known to a person of ordinary skill in the art may be employed. Different types of biasing elements are discussed in greater detail in related application entitled “Dynamic rod” bearing application Ser. No. 12/154,540 filed on May 23, 2008 and herein incorporated by reference in its entirety.

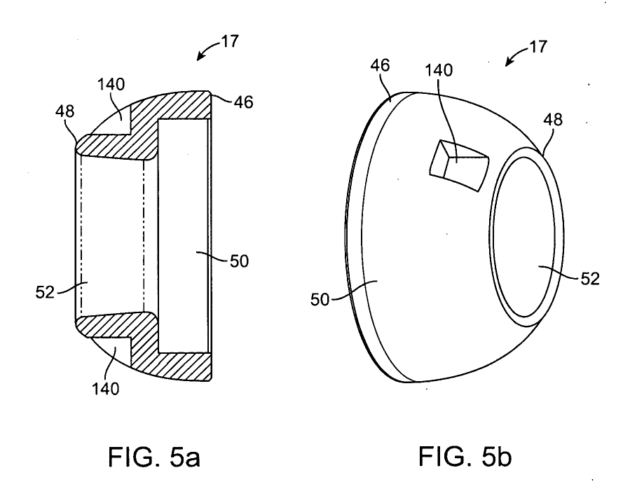

Turning now to FIGS. 5a and 5b, there is shown a retainer 17 having a first end 46 and a second end 48 according to the present invention. The retainer 17 is generally cylindrical and sleeve-like in shape and has a bore opening to and extending between the first and second ends 46, 48. The retainer 17 is configured to encompass at least a portion of the first rod portion 12 and at least a portion of the second rod portion 14 as shown in FIG. 2c. Accordingly, the bore defines a first receiving portion 50 at the first end 46 configured to receive therein at least a portion of the first rod portion 12 and, in particular, configured to receive the collar 34 of the first rod portion 12 as shown in FIG. 2c. The bore also defines a second receiving portion 52 at the second end 48 that is configured to receive therein at least a portion of the second rod portion 14. The retainer 17 forms a constriction such that the second end 48 has a smaller diameter relative to the diameter of the retainer at the first end 46. The interior surface of the retainer 17 substantially corresponds to the geometry being received within the retainer 17 with an abutment created at the intersection of the first and second receiving portions 50 and 52. The retainer 17 also includes apertures or notches 140 for orienting the rod 10 with an insertion instrument during installation.

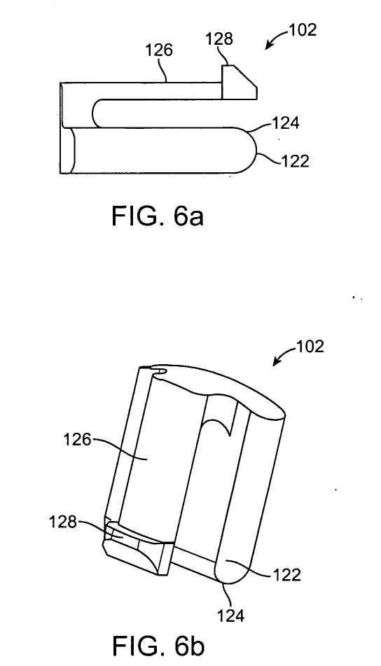

Turning now to FIGS. 6a and 6b, there is shown a dynamic lock 102 according to the present invention. The dynamic lock 102 includes a pusher 122 having a curved end 124 cantilevered to a spring lock portion 126 having a hook 128 at the end. The cantilevered end 124 is the end that engages an instrument configured to actuate the dynamic lock 102 through the dynamic lock engaging aperture 106.

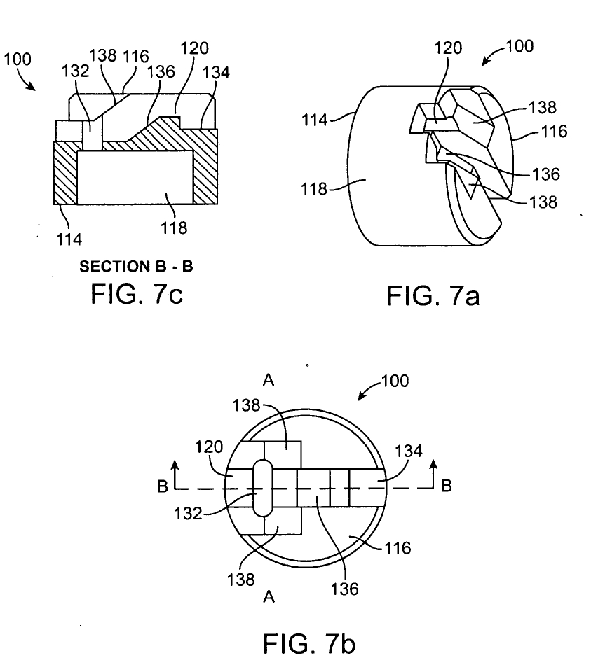

Turning now to FIGS. 7a, 7b and 7c, there is shown the dynamic slide 100 of the present invention. The dynamic slide 100 includes a first end 114 and a second end 116. A bore opening at the first end 114 defines a bias element receiving portion 118 configured to receive at least a portion of the bias element 16 therein. At the second end 116, there is a dynamic lock receiving portion 120 configured to engage with the dynamic lock 102. The dynamic lock receiving portion 120 includes an unlocked well 132 in which the hook 128 of the spring lock portion 126 resides when in an unlocked position and a locked well 134 in which the hook 128 of the spring lock portion 126 resides when in a locked position. The dynamic lock receiving portion 120 also includes a spring lock portion ramp 136 against which the spring lock portion 126 rides in going from the unlocked position to the locked position and vice versa. The dynamic lock receiving portion 120 also includes a pusher ramp 138 against which the pusher 122 rides in going from an unlocked position to a locked position.

Referring back to FIGS. 1 through 7, the assembly of the dynamic rod 10 will now be discussed. The dynamic lock 102 is placed in the dynamic lock receiving portion 120 of the locking slide 100 and inserted into the locking slide receiving portion 104 of the first rod portion 12. The bias element 16 is inserted into the bias element receiving portion 112 of the second rod portion 14. The second rod portion 14 together with the bias element 16 are inserted into the receiving portion 30 of the first rod portion 12 such that the bias element 16 is disposed inside the bias element receiving portion 118 of the locking slide 100. The retainer 17 placed over the shaft of the second rod portion 14 from the second end 38 and passed toward the first end 36 such that the engaging portion 40 resides in the first rod portion 12 and the collar 43 is received in the first receiving portion 50 of the retainer 17. The retainer 17 is connected to the first rod portion 12 by a laser weld or an e-beam weld or other suitable means such that the second rod portion 14 and bias element 16 are captured by the retainer 17 constriction and retained within the retainer 17 and the first rod portion 12 such that the second rod portion 14 is capable of movement relative to the retainer 17 and the first rod portion 12. In particular, the second rod portion 14 is capable of displacement from the longitudinal axis and/or movement along the longitudinal axis relative to the retainer 17 and the first rod portion 12. The bias element 16 may also be connected to locking slide 100 via a laser or e-beam weld.

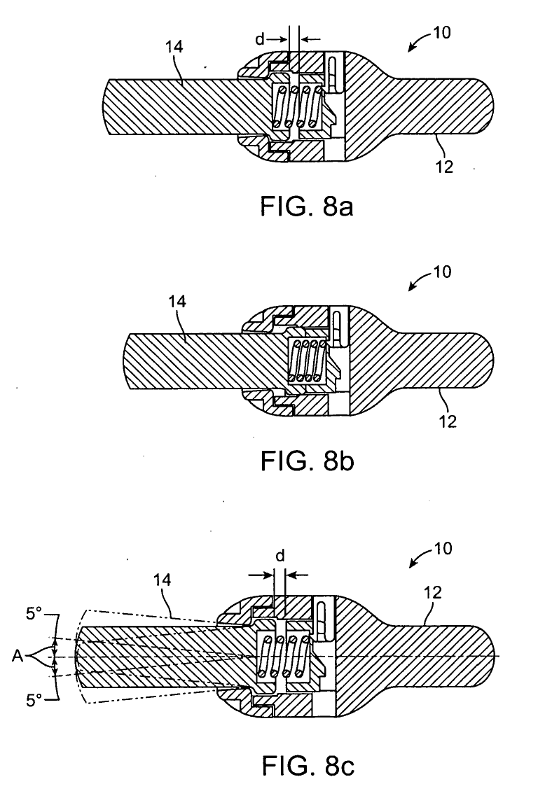

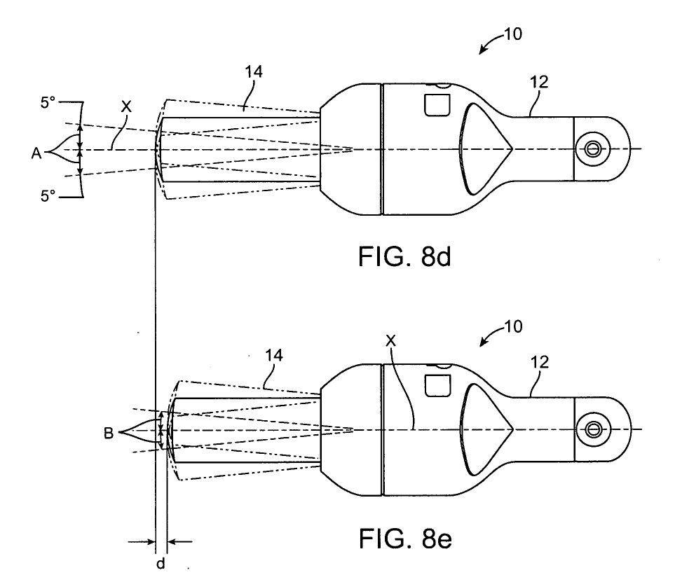

Turning now to FIGS. 8a-8e, movement of the second rod portion 14 relative to the first rod portion 12 will be discussed. Movement of the second rod portion 14 relative to the first rod portion 12 along the longitudinal axis such that the rod 10 is moving from a normal position into extension is shown in FIGS. 8a and 8b wherein FIG. 8a shows the rod 10 fully extended by a distance “d” and FIG. 8b shows the rod 10 in a fully compressed condition. Distance “d” is approximately 1 millimeter and preferably approximately between 0 and 10 millimeters and more preferably between 0 and 5 millimeters. Travel of the second rod portion 14 relative to the first rod portion 12 is biased by the bias element 16 in extension in one variation of the invention, in compression in another variation of the invention and in both extension and compression in a yet another variation of the invention. In response to such extension, the bias element 16 exerts a force inwardly to return the second rod portion 14 into a normal position. In another variation, the bias element 16 exerts a force outwardly to return the second rod 14 portion relative to the first rod portion 12 when compressed to a distance less than the maximum distance “d”. When fully extended from the first rod portion 12, the second rod portion 14 defines a distance “d” between the end of the first end 36 of the second rod portion 14 and the first end 114 of the locking slide 100. This distance “d” defines in part the extent of movement along the longitudinal axis of the second rod portion 14 relative to the first rod portion 12. In one variation, the distance “d” is approximately one or two millimeters. Distance “d” may be customized according to surgeon preference or be selected to be a suitable distance.

After the dynamic rod 10 is assembled, it is ready to be implanted within a patient and be connected to anchors planted in pedicles of adjacent vertebral bodies preferably in a manner such that the first rod portion 12 of the dynamic rod 10 is oriented cephalad and connected to the upper anchor and the second rod portion 14 is placed caudad and connected to the lower anchor. Because the first rod portion 12 includes an anchor connecting portion 22 configured such that connection with the anchor does not result in the rod extending cephalad beyond the anchor, this orientation and configuration of the dynamic rod is advantageous particularly because it avoids impingement of adjacent anatomy in flexion or in extension of the spine of the patient.

In an alternative variation, the dynamic rod 10 is implanted into the patient such that the first rod portion 12 is oriented caudad and the second rod portion 14 is oriented cephalad. In this variation, the second rod portion 14 includes an anchor connecting portion 44 that is partially spherical in shape and includes oppositely disposed outwardly extending pins 54 for engaging slots formed in the upper anchor to allow the dynamic rod 10 to pivot about pins 54 when connected to the anchor. Of course any connection means is permitted and not limited to a pin-slot engagement. The anchor connecting portion 44 may also include oppositely disposed flat areas 56 as described above. The second rod portion 14 of the dynamic rod 10 is oriented cephalad and connected to the upper anchor and the first rod portion 12 is placed caudad and connected to the lower anchor. Because the second rod portion 14 includes an anchor connecting portion 44 configured such that connection with the anchor does not result in excessive rod extending cephalad beyond the anchor, this orientation and configuration of the dynamic rod is advantageous particularly because it avoids impingement of adjacent anatomy in flexion or in extension of the spine of the patient.

Therefore, it is noted that the preferred implantation method and preferred orientation of the dynamic rod 10 is such that there is minimal or substantially no “overhanging” rod extending cephalad beyond the upper anchor. Such orientation is achieved by the orientation of the rod during implantation as well as by the configuration of the anchor connecting portion 22, 44 of either one or both of the first rod portion 12 and second rod portion 14 such that the anchor connecting portion 22, 44 is configured such that there is substantially no or little overhang beyond the anchor.

The implanted dynamic rod and anchor system fixes the adjacent vertebral bodies together in a dynamic fashion providing immediate postoperative stability and support of the spine. Still referencing FIGS. 8a-8e, FIGS. 8c and 8d illustrate displacement from the longitudinal axis of the second rod portion 14 relative to the first rod portion 14 by an angle “A” while the second rod portion 14 is also longitudinally in extension relative to the first rod portion 12 by a distance “d”.

Angle “A” is approximately between zero and ten degrees, preferably approximately five degrees with respect to the longitudinal axis “x” in a polyaxial direction from the longitudinal axis “x”. FIG. 8e shows the second rod portion 14 displaced from the longitudinal axis “x” in any polyaxial direction relative to the longitudinal axis “x” by an angle “B” while the second rod portion is also longitudinally in compression relative to the first rod portion 12 by a distance “d”. Angle “B” is approximately between zero and ten degrees, and preferably approximately five degrees with respect to the longitudinal axis “x”.

Hence, FIGS. 8a-8e illustrate that the dynamic rod allows for movement described by a polyaxial displacement from the longitudinal axis as well as movement along the longitudinal axis in extension or compression alone or in combination with polyaxial motion allowing the rod to carry some of the natural flexion and extension moments that the spine is subjected to. Substantial polyaxial rotation of the second rod portion relative to the first rod portion is within the scope of motion of the dynamic rod. However, rotation of the second rod portion 14 relative to the first rod portion 12 may be constrained by a squared first end 36 of the second rod portion 14 inserted into a conformance formed by the bias element receiving portion 118 of the locking slide 100. This feature controls rotation and provides torsional strength and resistance.

In one variation, the bias element 16 is a compression spring that becomes shorter when axially loaded and acts as an extension mechanism such that when disposed in the assembled dynamic rod 10 and axially loaded, the bias element 16 exerts a biasing force pushing the first rod portion 12 and the second rod portion 14 apart. In one variation, the bias element 16 is configured such that it exerts a biasing force pushing the first rod portion 12 and second rod portion 14 apart by the maximum degree permitted by the dynamic rod configuration such that when longitudinally loaded the second rod portion 14 will move inwardly towards the first rod portion 12 and the bias element will tend to push the second rod portion 14 outwardly.

In another variation, the bias element 16 is a coil configured to not exhibit spring-like characteristics when loaded along the longitudinal axis. Instead, the coil serves a stabilizer for loads having a lateral force component, in which case the lateral biasing is provided by the bias element.

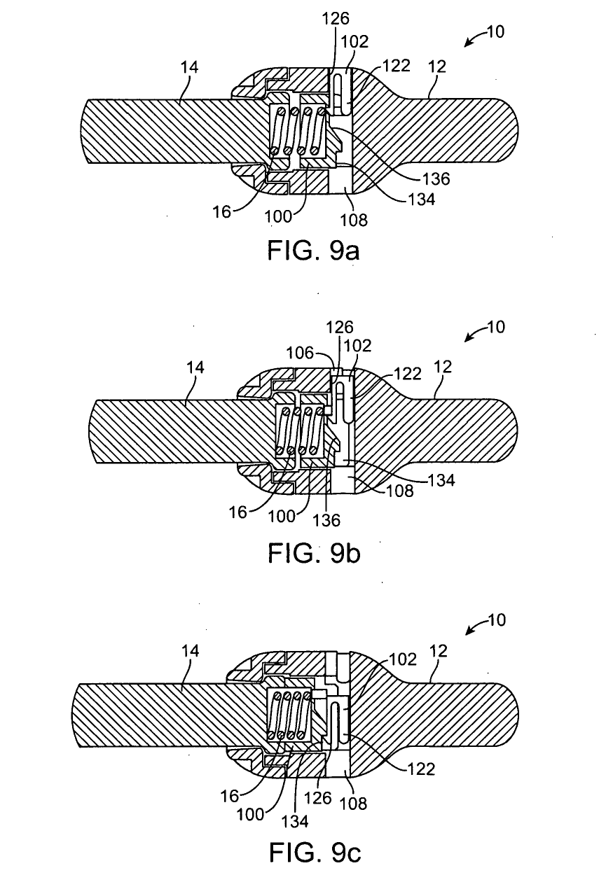

Another advantageous feature of the dynamic rod 10 according to the present invention is that it can be locked. In one variation, the dynamic rod 10 according to the present invention can be locked in extension. Turning now to FIGS. 9a, 9b and 9c, there is shown a rod 10 according to the present invention. FIG. 9a illustrates the rod 10 in an unlocked configuration in which the second rod portion 14 is free to translate longitudinally as well as angulate polyaxially. In FIG. 9b, the dynamic lock 102 is engaged through the dynamic lock engaging aperture 106 by an instrument (not shown) such that the pusher 122 of the dynamic lock 102 contacts the pusher ramp 138 of the locking slide 100 and further advancement of the dynamic lock 102 results in the pusher 102 riding the pusher ramp 138 pushing the locking slide 100 away from the first rod portion 12. Simultaneously, the spring lock portion 126 of the dynamic lock contacts the spring lock portion ramp 136 and further advancement of the dynamic lock 102 results in the spring lock portion 126 riding the spring lock portion ramp 136 until the hook 128 springs into the locked well 134 into a locked configuration as shown in FIG. 9c. When in the locked configuration, the second rod portion 14 is fully extended and hence, incapable of further extension along the longitudinal axis. In one variation, when in the locked configuration, the second rod portion 14 is permitted to angulate and in another variation, the second rod portion 14 is also locked from angulation as shown in FIG. 9c. In another variation, the locked position is the fully extended and non-angulated position. In another variation, the locked position is an angulated position. In another variation, the distance of the first rod portion from the second rod portion is locked in place. In another variation, the first and second rod portions are lockable in a fully compressed orientation. In another variation, the first and second rod portions are lockable in a fully compressed orientation or extended configuration or at any distance of longitudinal extension which still permitting angulation to take place and in another variation the angulation is also locked. The dynamic rod 10 may be unlocked by insertion of an instrument into the dynamic lock release aperture 108 to push the dynamic lock 102 into an unlocked configuration. In one variation, only the Hence, this invention sets forth a dynamic rod 10 that is capable of being locked and unlocked according to surgeon preference. In some cases, individual rods in a spinal fixation system require individual adjustment to fine-tune the installation based on patient anatomy or surgeon preference and the present invention addresses this need.

The disclosed devices or any of their components can be made of any biologically adaptable or compatible materials including PEEK, PEK, PAEK, PEKEKK or other polyetherketones. Materials considered acceptable for biological implantation are well known and include, but are not limited to, stainless steel, titanium, tantalum, combination metallic alloys, various plastics, polymers, resins, ceramics, biologically absorbable materials and the like. Any components may be also coated with various coatings or made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation as well as with anti-microbial materials. Further, a surface of any of the implants may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone ingrowth or establish a mineralized connection between the bone and the implant, and reduce the likelihood of implant loosening. Lastly, any assembly or its components can also be entirely or partially made of a shape memory material or other deformable material. Of course, the second rod portion 14 and/or first rod portion 12 may be slightly curved to provide an overall curved rod 10 for conforming to patient anatomy and, of course, the rod 10 may be substantially straight.

From the above, it is evident that the present invention can be used to relieve pain caused by spinal stenosis in the form of, by way of example only, central canal stenosis or foraminal stenosis, degenerative disc disease, spondylolisthesis, spinal deformaties, fracture, pseudarthrosis and tumors.

All publications mentioned herein are incorporated herein by reference to disclose and describe the methods and/or materials in connection with which the publications are cited. The preceding illustrates the principles of the invention. It will be appreciated that those skilled in the art will be able to devise various arrangements which, although not explicitly described or shown herein, embody the principles of the invention and are included within its spirit and scope.

We claim:

1. A dynamic rod implantable in a spine comprising:
a first rod portion having a first engaging portion at a first end;
a second rod portion having a second engaging portion at a first end, the first and second rod portions connected to each other at the first and second engaging portions such that the first rod portion and second rod portion are capable of relative motion; and
a lock configured to lock said relative motion.

2. The dynamic rod of claim 1 wherein the lock is reversible.

3. The dynamic rod of claim 1 further including a bias element disposed between the first rod portion and the second rod portion.

4. The dynamic rod of claim 1 wherein said relative motion is angulation of the first rod portion relative to the second rod portion or longitudinal translation of the first rod portion relative to the second rod portion.

5. The dynamic rod of claim 1 wherein said relative motion is angulation of the first rod portion relative to the second rod portion and longtudinal translation of the first rod portion relative to the second rod portion.

6. The dynamic rod of claim 1 wherein the lock includes a spacer movable to a locked position between the first and second rod portions to arrest said relative motion.

7. The dynamic rod of claim 6 wherein the lock includes a ramp portion configured to provide ramp surface for the spacer to move against into a locked position.

8. The dynamic rod of claim 7 wherein the spacer and ramp portion are located in the first engaging portion and the second rod portion is nested inside the first engaging portion; wherein when in the locked position the ramp portion abuts the first end of the second rod portion and the spacer abuts the ramp portion.

9. The dynamic rod of claim 1 further including an aperture for percutaneously engaging said lock.

10. A dynamic rod implantable in a spine comprising:
a first rod portion coupled to a second rod portion and configured such that movement of one rod portion with respect to the other rod portion is lockable in position by a lock.

11. The dynamic rod of claim 10 wherein the movement is longitudinal translation or angulation of one rod portion with respect to the other rod portion.

12. The dynamic rod of claim 10 wherein the movement of one rod portion with respect to the other rod portion is reversibly lockable in position by the lock.

13. The dynamic rod of claim 10 wherein the longitudinal translation of one rod portion with respect to the other rod portion is lockable by the lock while permitting the angulation of one rod portion with respect to the other rod portion.

14. The dynamic rod of claim 10 wherein the angulation of one rod portion with respect to the other rod portion is lockable by the lock while permitting the longitudinal translation of one rod portion with respect to the other rod portion.

15. The dynamic rod of claim 10 wherein the first rod portion is lockable at any distance from the second rod portion.

16. The dynamic rod of claim 10 wherein the rod is lockable in position such that the first rod portion is fully extended from the second rod portion.

17. The dynamic rod of claim 10 wherein the rod is lockable in position such that the first rod portion is angled with respect to the second rod portion.

18. The dynamic rod of claim 10 wherein the rod is lockable in position such that the first rod portion is fully compressed towards the second rod portion.

19. The dynamic rod of claim 10 wherein the lock comprises an element movable transversely to the longitudinal axis of the rod to a locked position.

20. The dynamic rod of claim 10 further including a spring disposed between the first and second rod portions.
